(19) United States
(12) Patent Application Publication
(10) Pub. No.: US 2020/0100688 A1
(43) Pub. Date: Apr. 2, 2020

(54) PATIENT MONITOR DEVICE

(71) Applicant: NIHON KOHDEN CORPORATION, Tokyo (JP)

(72) Inventor: Shiro MURATA, Tokorozawa-shi (JP)

(21) Appl. No.: 16/582,274

(22) Filed: Sep. 25, 2019

(30) Foreign Application Priority Data
Sep. 28, 2018 (JP) 2018-183366

(51) Int. Cl. A61B 5/021; G16H 40/63; A61B 5/00

(52) U.S. Cl. CPC A61B 5/02141; G16H 40/63; A61B 5/14551; A61B 5/0022; A61B 5/002; A61B 5/742

(57) ABSTRACT

A patient monitor device configured to display vital information of a living body includes: a display; and circuitry configured to individually display at least two types of the vital information of the living body on a plurality of display regions of the display. Each of the display regions includes: a numerical value display portion for displaying measured vital information of the living body as a numerical value; and a tab portion for notifying of completion of the measurement after the measurement of the vital information is completed, and tab portions of respective display regions are arranged in a line along an array of respective numerical value display portions.

### CROSS-REFERENCE TO RELATED APPLICATIONS

This application is based on and claims priority under 35 USC 119 from Japanese Patent Application No. 2018-183366 filed on Sep. 28, 2018, the contents of which are incorporated herein by reference.

### TECHNICAL FIELD

The presently disclosed subject matter relates to a patient monitor device which can display completion of measurement of vital information of a living body, such as blood pressure, with high visibility.

### BACKGROUND

In related art, a patient monitor device which is called a bedside monitor is disposed on, for example, a bedside of a patient after surgery or a patient after catheter treatment so as to constantly monitor a change in a condition of the patient.

Meanwhile, a patient whose condition is recovering does not always need to be monitored through the bedside monitor, but it is necessary to periodically check vital information of the patient, such as blood pressure. Thus, in recent years, a medical worker such as a nurse who periodically walks around a hospital room uses a patient monitor device which is called a spot check monitor, so as to measure vital information of a patient and to transmit the vital information to an electronic medical record.

However, since vital information of a plurality of patients is measured while the medical worker walks busily around hospital rooms, it is difficult for the medical worker to recognize vital information measurement of which patient is completed only by looking at display on the patient monitor device.

In the patient monitor device, vital information whose measurement is completed and vital information during measurement are displayed as numerical values. Thus, displayed numerical values must be seen carefully to know whether the measurement is completed or ongoing, and thus it cannot be grasped sensually at a glance.

Therefore, an object of the presently disclosed subject matter is to provide a patient monitor device which can display completion of measurement of vital information of a living body, such as blood pressure, with high visibility.

### SUMMARY

A patient monitor device to achieve the object is configured to display vital information of a living body. The patient monitor device includes a display; and circuitry configured to individually display at least two types of the vital information of the living body on a plurality of display regions of the display. Each of the display regions includes: a numerical value display portion for displaying measured vital information of the living body as a numerical value; and a tab portion for notifying of completion of the measurement after the measurement of the vital information is completed, and tab portions of respective display regions are arranged in a line along an array of respective numerical value display portions.

### BRIEF DESCRIPTION OF DRAWINGS

FIG. 1 is an external view of a patient monitor device of Embodiment 1;

FIG. 2 is a block diagram of a controller of the patient monitor device of Embodiment 1;

FIG. 3 is an operation flowchart of the patient monitor device of Embodiment 1;

FIG. 4 illustrates a display of the patient monitor device of Embodiment 1 before measurement:

FIG. 5 illustrates the display of the patient monitor device of Embodiment 1 when the measurement is completed;

FIG. 6 illustrates the display of the patient monitor device of Embodiment 1 after the measurement is completed;

FIG. 7 illustrates the display of the patient monitor device of Embodiment 1 when a part of the measurement is not completed;

FIG. 8 is an operation flowchart of a patient monitor device of Embodiment 2;

FIG. 9 illustrates a display of the patient monitor device of Embodiment 2 before measurement;

FIG. 10 illustrates the display of the patient monitor device of Embodiment 2 when the measurement is completed;

FIG. 11 illustrates the display of the patient monitor device of Embodiment 2 after the measurement is completed; and

FIG. 12 illustrates the display of the patient monitor device of Embodiment 2 when a part of the measurement is not completed.

### DESCRIPTION OF EMBODIMENTS

Next, embodiments of a patient monitor device of the presently disclosed subject matter will be described separately in Embodiment 1 and Embodiment 2.

### Embodiment 1

(Configuration of Patient Monitor Device)

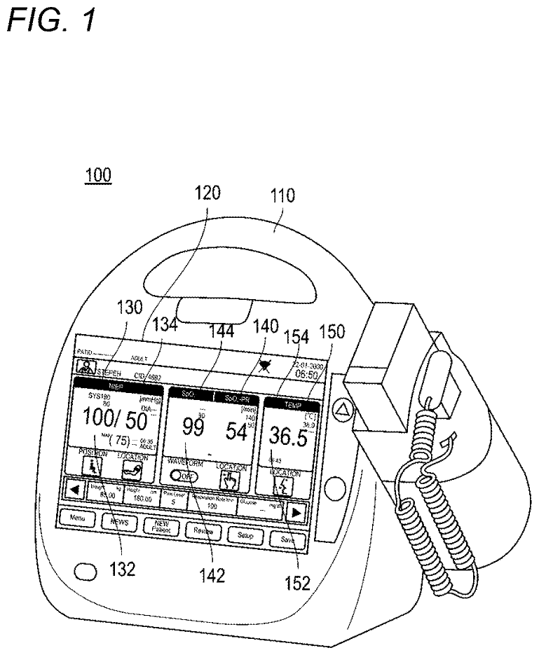

FIG. 1 is an external view of a patient monitor device of Embodiment 1. A patient monitor device 100 can include a carrying handle 110 so that a medical worker such as a nurse can easily carry the patient monitor device 100 around hospital rooms. The patient monitor device 100 can include, on a front side thereof, a display 120 that displays vital information of a patient, such as non-invasive blood pressure (NIBP), arterial oxygen saturation (SpO₂), pulse rate (SpO₂-PR), and body temperature (TEMP).

A plurality of display regions 130, 140 and 150, in which at least two types of the vital information of the patient are individually displayed, are formed in the display 120 of the patient monitor device 100. Specifically, the display region 130 displays the non-invasive blood pressure (NIBP) of the patient, the display region 140 displays the arterial oxygen saturation (SpO₂) and the pulse rate (SpO₂-PR) of the patient, and the display region 150 displays the body temperature (TEMP) of the patient.

Respective display regions 130, 140 and 150 can include: numerical value display portions 132, 142 and 152 that display the measured vital information of the patient as numerical values; and tab portions 134, 144 and 154 that notify completion of measurement after the measurement of the vital information is completed. The tab portions 134, 144 and 154 of the respective display regions 130, 140 and 150 are arranged in a line along an array of the respective numerical value display portions 132, 142 and 152.

The tab portions 134, 144, 154 extend in a lateral width direction of the respective display regions 130, 140, and 150 on upper sides of the numerical value display portions 132, 142, and 152 in respective display regions 130, 140 and 150. In the present embodiment, lateral widths of the tab portions 134, 144, and 154 are substantially the same as lateral widths of the respective display regions 130, 140, and 150. In the present embodiment, the tab portions 134, 144, and 154 are provided on the upper sides of the numerical value display portions 132, 142, and 152, but the tab portions 134, 144, and 154 may also be provided on lower sides of the numerical value display portions 132, 142 and 152. The lateral widths of the tab portions 134, 144, and 154 may also be shorter than the lateral widths of the display regions 130, 140, and 150. Tabs or display parts may be additionally provided on upper, lower, left, and right sides of the tab portions 134, 144, and 154 to indicate that the measurement is completed.

The tab portions 134, 144, and 154 are lighted or blinked after the measurement of the vital information is completed. The completion of the measurement of the vital information is notified in this way. Types of the measured vital information are displayed on the tab portions 134, 144, and 154 by characters. Specifically, if the measured vital information in the tab portion 134 is the non-invasive blood pressure of the patient, the characters “NIBP” are displayed; if the measured vital information in the tab portion 144 is the arterial oxygen saturation and the pulse rate of the patient, the characters “Spa,” and “SpO₂-PR” are displayed; if the measured vital information in the tab portion 154 is the body temperature of the patient, the characters “TEMP” are displayed.

The characters of the tab portions 134, 144, and 154 are lighted or blinked before the measurement of the respective vital information is completed. Meanwhile, after the measurement is completed, portions other than the characters of the tab portions 134, 144, and 154 are lighted or blinked. In the present embodiment, colors of respective numbers displayed on the numerical value display portions 132, 142, and 152 of the respective display regions 130, 140, and 150 are the same as colors of the respective tab portions 134, 144, and 154.

For example, if the color of the characters displayed on the numerical value display portion 132 of the display region 130 is red, the color of the tab portion 134 of the display region 130 (including characters of the tab portion) is also red. If the color of the characters displayed on the numerical value display portion 142 of the display region 140 is blue, the color of the tab portion 144 of the display region 140 (including characters of the tab portion) is also blue. Further, if the color of the characters displayed on the numerical value display portion 152 of the display region 150 is orange, the color of the tab portion 154 of the display region 150 (including characters of the tab portion) is also orange.

The colors of the respective numbers displayed on the numerical value display portions 132, 142, and 152 of the respective display regions 130, 140, and 150 may also be different from the colors of the respective tab portions 134, 144, and 154. The colors of the tab portions 134, 144, and 154 are not limited to the colors exemplified above, and other colors may also be used. The colors of the tab portions 134, 144, and 154 may be the same color or different colors as long as the completion of the measurement of the vital information can be sensed at a glance.

Display area of each of the display regions 130, 140 and 150 is changed in accordance with the number of the types of the measured vital information. In the present embodiment, since the number of the types of the measured vital information is three, as illustrated in FIG. 1, the display 120 is divided into three parts as the three display regions 130, 140, and 150 displayed in the display 120. As described below, in a case where the number of the types of the measured vital information is two, the display 120 is divided into two parts as two display regions displayed in the display 120. Same or similarly, in a case where the number of the types of the measured vital information is four or more, the display 120 is divided into four parts or more to perform display in the display 120.

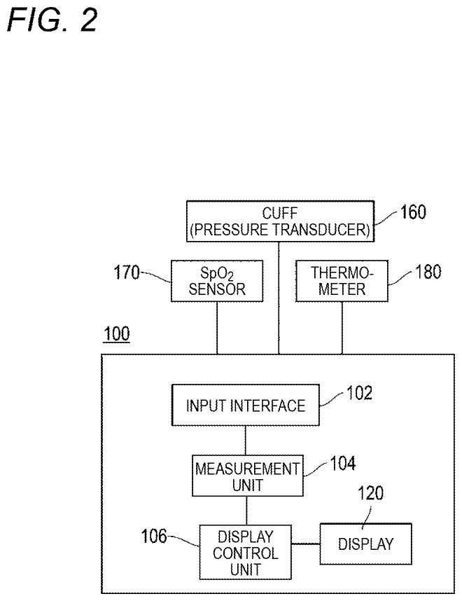

FIG. 2 is a block diagram of a controller of the patient monitor device 100 of Embodiment 1. The patient monitor device 100 can include an input interface 102, a measurement unit 104, a display controller 106, and the display 120.

The input interface 102 is connected with a cuff (pressure transducer) 160 wrapped around an arm of the patient to measure the non-invasive blood pressure of the patient, an SpO₂ sensor 170 mounted on a fingertip the patient to measure the arterial oxygen saturation “SpO₂” and the pulse rate “SpO₂-PR” of the patient, and a thermometer 180 that measures the body temperature of the patient, and inputs these vital information of the patient.

The measurement unit 104 inputs the vital information input via the input interface 102, such as the non-invasive blood pressure of the patient, the arterial oxygen saturation “SpO₂” and the pulse rate “SpO₂-PR” of the patient, and the body temperature of the patient, and measures a maximum value and a minimum value of the non-invasive blood pressure, the arterial oxygen saturation “SpO₂” and the pulse rate “SpO₂-PR” and the body temperature.

The display controller 106 controls the display 120 to display the maximum value and the minimum value of the non-invasive blood pressure, the arterial oxygen saturation “SpO₂” and the pulse rate “SpO₂-PR”, and the body temperature, which are measured by the measurement unit 104.

The display 120 displays the vital information such as the maximum value and the minimum value of the non-invasive blood pressure, the arterial oxygen saturation “SpO₂” and the pulse rate “SpO₂-PR” and the body temperature in a display mode as illustrated in FIG. 1 in the patient monitor device 100.

(Operation of Patient Monitor Device)

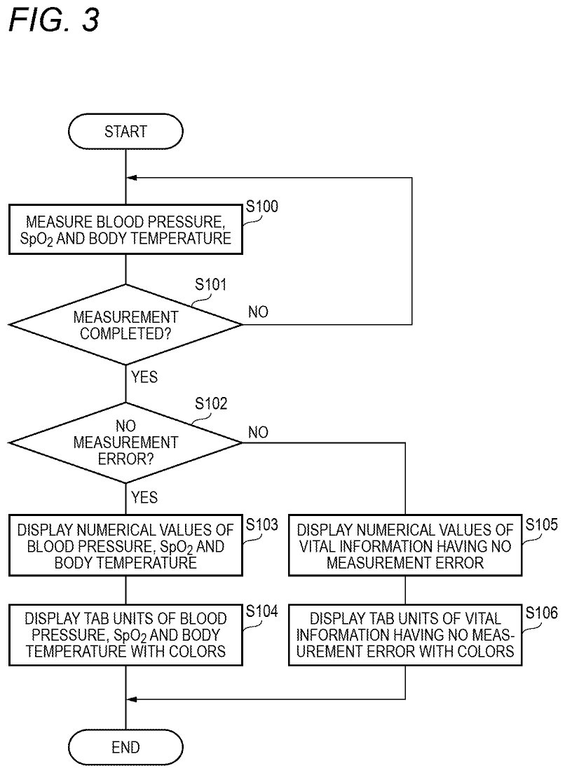

FIG. 3 is an operation flowchart of the patient monitor device 100 of Embodiment 1.

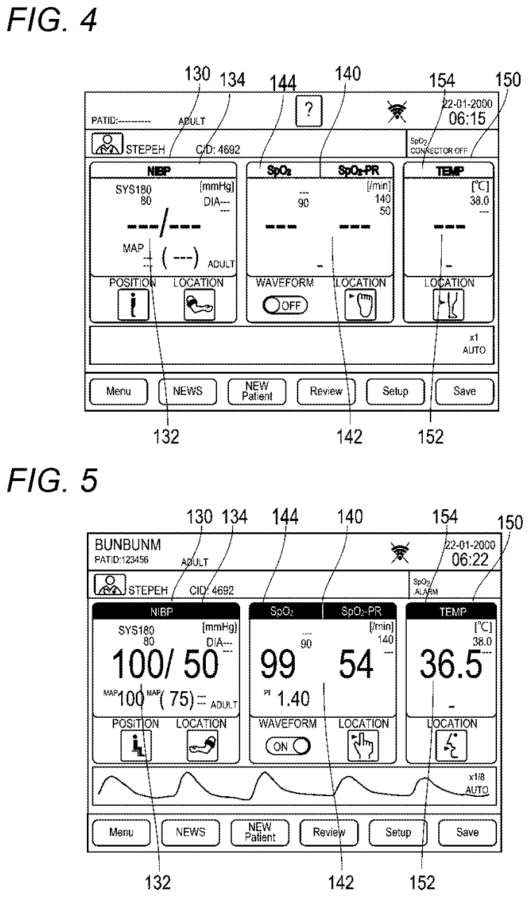

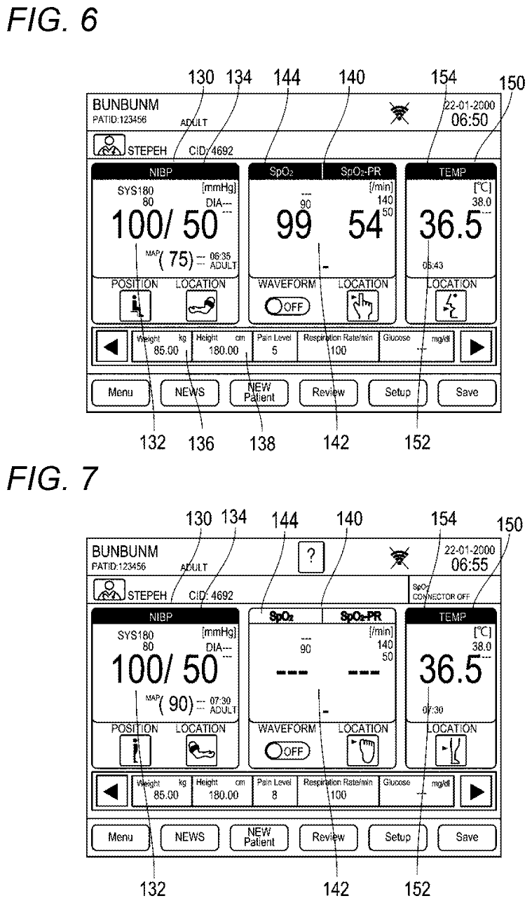

FIG. 4 illustrates the display 120 of the patient monitor device 100 of Embodiment 1 before measurement. FIG. 5 illustrates the display 120 of the patient monitor device 100 of Embodiment 1 when the measurement is completed. FIG. 6 illustrates the display 120 of the patient monitor device 100 of Embodiment 1 after the measurement is completed. FIG. 7 illustrates the display 120 of the patient monitor device 100 of Embodiment 1 when a part of the measurement is not completed. Next, an operation of the patient monitor device 100 will be described with reference to FIGS. 4 to 7.

First, a medical worker, such as a nurse, wraps the cuff 160 around the arm of the patient, mounts the SpO₂ sensor 170 on the fingertip of the patient, places the thermometer 180 below the arm of the patient so as to be sandwiched, and starts measurement of the vital information of the patient. Display of the display 120 of the patient monitor device 100 before the measurement is as illustrated in FIG. 4. That is, before the measurement, no numerical value is displayed on the respective numerical value display portions 132, 142, and 152 of the respective display regions 130, 140, and 150, and the characters indicating the types of the vital information are displayed with predetermined colors in the tab portions 134, 144, and 154, respectively. The characters indicating the types of the vital information are lighted or blinked before the measurement of the vital information is completed.

The measurement unit 104 of the patient monitor device 100 inputs, via the input interface 102 respectively, the non-invasive blood pressure of the patient received from the pressure transducer of the cuff 160, the arterial oxygen saturation “SpO₂” and the pulse rate “SpO₂-PR” received from the SpO₂ sensor 170, and the body temperature of the patient received from the thermometer 180. Then, the measurement unit 104 measures, from the input vital information, the maximum value and the minimum value of the non-invasive blood pressure, the arterial oxygen saturation “SpO₂” and the pulse rate “SpO₂-PR” and the body temperature of the patient (S100).

Next, the measurement unit 104 determines whether measurement of all the vital information is completed (S101). If the measurement is not completed (S101: NO), the measurement unit 104 continues the measurement of these vital information. If the measurement is completed (S101: YES), the measurement unit 104 determines whether there is no measurement error in the measured vital information (S102).

If there is no measurement error in the measurement of all of the vital information (S102: YES), the measurement unit 104 instructs the display controller 106 to display, on the display 120, the maximum value and the minimum value of the measured non-invasive blood pressure, the arterial oxygen saturation “SpO₂”and the pulse rate “SpO₂-PR”, and the body temperature of the patient. The display 120 displays, by numerical values, the maximum value and the minimum value of the non-invasive blood pressure, and values of each of the arterial oxygen saturation “SpO₂” and the pulse rate “SpO₂-PR”, and the body temperature of the patient in accordance with the instruction of the display controller 106 (S103).

Next, the display 120 displays, with predetermined colors respectively, the tab portions provided on the numerical value display portions of the maximum value and the minimum value of the non-invasive blood pressure, the arterial oxygen saturation “SpO₂” and the pulse rate “SpO₂-PR”, and the body temperature of the patient in accordance with the instruction of the display controller 106 (S104).

The display of the display 120 of the patient monitor device 100 when the measurement is completed is as illustrated in FIG. 5. That is, the numerical values of the respective measured vital information are displayed on the respective numerical value display portions 132, 142, and 152 of the respective display regions 130, 140, and 150, and portions other than the characters of the tab portions 134, 144, and 154 are colored respectively with predetermined colors and are lighted or blinked. Since the tab portions 134, 144, and 154 are arranged in a line along the array of the respective numerical value display portions 132, 142, and 152, the completion of the measurement of the vital information of the patient is displayed with good visibility, and the completion of the measurement of all vital information can be sensed at a glance.

After the measurement is completed, the medical worker can manually input the vital information of the patient. For example, as illustrated in FIG. 6, a weight 85.00 of the patient can be manually input into a weight column 136 and a height 180.00 of the patient can be manually input into a height column 138. When the input operation is completed in this way, the vital information whose measurement is completed is deleted, and the tab portions 134, 144, and 154 are not displayed with colors. Accordingly, when starting measurement of vital information of a new patient, the medical worker can sense the measurement at a glance without misunderstanding the measurement as already-completed measurement.

Meanwhile, if there is a measurement error in the measurement of any vital information (S102: NO), the measurement unit 104 instructs the display controller 106 to display, on the display 120, the vital information which has no measurement error among the maximum value and the minimum value of the measured non-invasive blood pressure, the arterial oxygen saturation “SpO₂” and the pulse rate “SpO₂-PR”, and the body temperature of the patient. The display 120 displays, by numerical values, the vital information which has no measurement error, for example, the maximum value and the minimum value of the non-invasive blood pressure, and a value of the body temperature of the patient in accordance with the instruction of the display controller 106 (S105).

Next, the display 120 displays, with predetermined colors respectively, the tab portions of the vital information which has no measurement error provided on the numerical value display portions of the maximum value and the minimum value of the non-invasive blood pressure, the arterial oxygen saturation “SpO₂” and the pulse rate “SpO₂-PR”, and the body temperature of the patient in accordance with the instruction of the display controller 106. For example, the tab portions 134 and 154 provided on the numerical value display portions of the maximum value and the minimum value of the non-invasive blood pressure and the body temperature of the patient are displayed with predetermined colors (S106).

The display of the display 120 of the patient monitor device 100 when a part of the measurement is not completed is as illustrated in FIG. 7. For example, when an error occurs in the measurement of the arterial oxygen saturation “SpO₂” and the pulse rate “SpO₂-PR” of the patient, such as forgetting to measure or being unable to measure, the numerical values of the respective measured vital information are displayed on the respective display regions 130 and 150, and portions other than the characters of the tab portions 134 and 154 are colored respectively with predetermined colors and are lighted or blinked. Therefore, no numerical value is displayed on the numerical value display portion 142 of the arterial oxygen saturation “SpO₂” and the pulse rate “SpO₂-PR”, and although the characters “SpO₂” and “SpO₂-PR” are displayed on the tab portion 144. the tab portion 144 is not displayed with the predetermined color. Since the tab portions 134, 144, and 154 are arranged in a line along the array of the respective numerical value display portions 132, 142, and 152, when a part of the measurement of the vital information of the patient is not completed, the fact that a part of the measurement is not completed can be sensed by the medical worker at a glance since tabs of the part are not displayed with colors.

### Embodiment 2

Next, a patient monitor device according to Embodiment 2 will be described. The Embodiment 2 is an embodiment in which two display regions are provided in a display of the patient monitor device.

(Configuration of Patient Monitor Device)

A configuration of the patient monitor device of the present embodiment is substantially the same as the configuration of the patient monitor device 100 of Embodiment 1. A difference is that the thermometer 180 is not connected to the patient monitor device 100, and vital information related to the body temperature of the patient is not displayed. Since the configuration of the patient monitor device of Embodiment 2 is substantially the same as the configuration of the patient monitor device 100 of Embodiment 1, reference numerals of components of the patient monitor device of Embodiment 2 are denoted by the same reference numerals as those of the components of the patient monitor device 100.

(Operation of Patient Monitor Device)

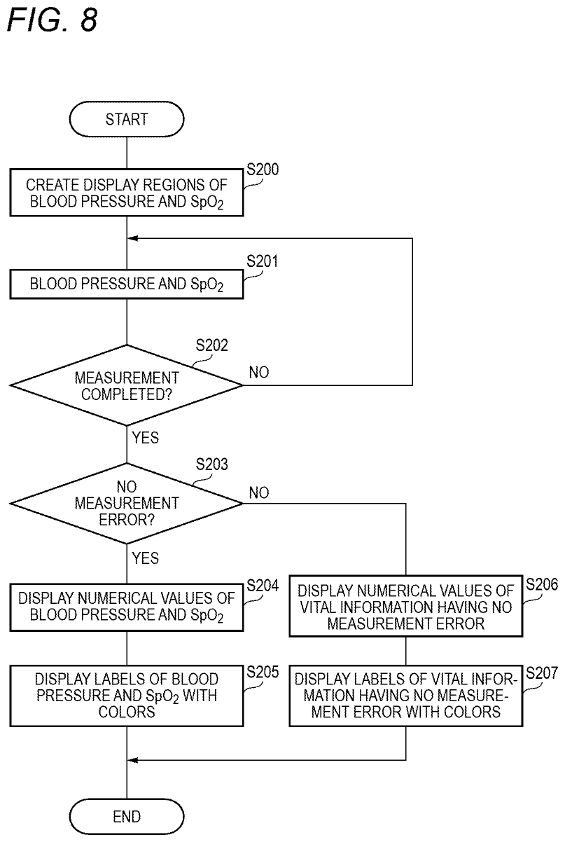

FIG. 8 is an operation flowchart of the patient monitor device of Embodiment 2.

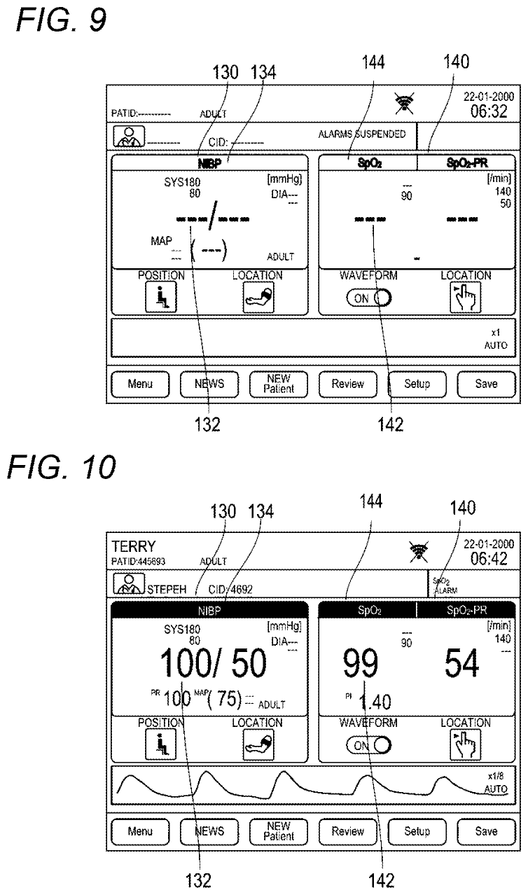

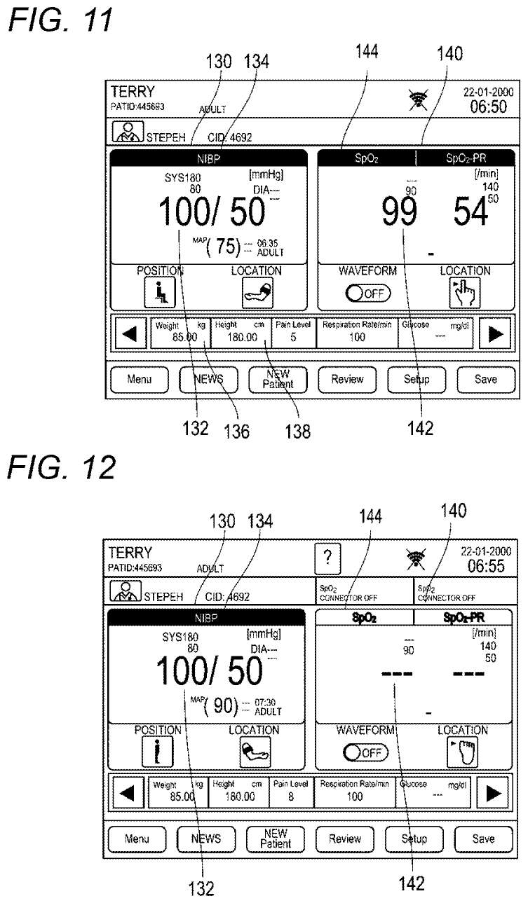

FIG. 9 illustrates a display of the patient monitor device of Embodiment 2 before measurement. FIG. 10 illustrates the display of the patient monitor device of Embodiment 2 when the measurement is completed. FIG. 11 illustrates the display of the patient monitor device of Embodiment 2 after the measurement is completed. FIG. 12 illustrates the display of the patient monitor device of Embodiment 2 when a part of the measurement is not completed. Next, the operation of the patient monitor device will be described with reference to FIGS. 9 to 12.

First, the display controller 106 approximately halves the display area of the display 120 in response to the fact that two devices, the cuff 160 and the SpO₂ sensor 170, are connected to the patient monitor device 100, and creates display regions of the non-invasive blood pressure, and the arterial oxygen saturation “SpO₂” and the pulse rate “SpO₂-PR”. For example, as illustrated in FIG. 9, the display regions of the non-invasive blood pressure, and the arterial oxygen saturation “SpO₂” and the pulse rate “SpO₂-PR” are created (S200).

Next, a medical worker, such as a nurse, wraps the cuff 160 around the arm of the patient, mounts the SpO₂ sensor 170 on the fingertip of the patient, and starts measurement of the vital information of the patient. Display of the display 120 of the patient monitor device 100 before the measurement is as illustrated in FIG. 9. That is, before the measurement, no numerical value is displayed on the respective numerical value display portions 132 and 142 of the respective display regions 130 and 140, and the characters indicating the types of the vital information are displayed with predetermined colors in the tab portions 134 and 144, respectively. The characters indicating the types of the vital information are lighted or blinked before the measurement of the vital information is completed.

The measurement unit 104 of the patient monitor device 100 inputs, via the input interface 102 respectively, the non-invasive blood pressure of the patient received from the pressure transducer of the cuff 160, and the arterial oxygen saturation “SpO₂” and the pulse rate “SpO₂-PR” received from the SpO₂ sensor 170. Then, the measurement unit 104 measures, from the input vital information, the maximum value and the minimum value of the non-invasive blood pressure, and the arterial oxygen saturation “SpO₂” and the pulse rate “SpO₂-PR” of the patient (S201).

Next, the measurement unit 104 determines whether measurement of all the vital information is completed (S202). If the measurement is not completed (S202: NO), the measurement unit 104 continues the measurement of these vital information. If the measurement is completed (S202: YES), the measurement unit 104 determines whether there is no measurement error in the measured vital information (S203).

If there is no measurement error in the measurement of all of the vital information (S203: YES), the measurement unit 104 instructs the display controller 106 to display, on the display 120, the maximum value and the minimum value of the measured non-invasive blood pressure, and the arterial oxygen saturation “SpO₂” and the pulse rate “SpO₂-PR” of the patient. The display 120 displays, by numerical values, the maximum value and the minimum value of the non-invasive blood pressure, and values of the arterial oxygen saturation “SpO₂” and the pulse rate “SpO₂-PR” of the patient in accordance with the instruction of the display controller 106 (S204).

Next, the display 120 displays, with predetermined colors respectively, the tab portions provided on the numerical value display portions of the maximum value and the minimum value of the non-invasive blood pressure, and the arterial oxygen saturation “SpO₂” and the pulse rate “SpO₂-PR” of the patient in accordance with the instruction of the display controller 106 (S205).

The display of the display 120 of the patient monitor device 100 when the measurement is completed is as illustrated in FIG. 10. That is, the numerical values of the respective measured vital information are displayed on the respective numerical value display portions 132 and 142 of the respective display regions 130 and 140, and portions other than the characters of the tab portions 134 and 144 are colored respectively with predetermined colors and are lighted or blinked. Since the tab portions 134 and 144 are arranged in a line along the array of the respective numerical value display portions 132 and 142, the completion of the measurement of the vital information of the patient is displayed with good visibility, and the completion of the measurement of all vital information can be sensed at a glance.

After the measurement is completed, the medical worker can manually input the vital information of the patient. For example, as illustrated in FIG. 11, the weight 85.00 of the patient can be manually input into the weight column 136 and the height 180.00 of the patient can be manually input into the height column 138. When the input operation is completed in this way, the vital information whose measurement is completed is deleted, and the tab portions 134 and 144 are not displayed with colors. Accordingly, when starting measurement of vital information of a new patient, the medical worker can sense the measurement at a glance without misunderstanding the measurement as already-completed measurement.

Meanwhile, if there is a measurement error in the measurement of any vital information (S203: NO), the measurement unit 104 instructs the display controller 106 to display, on the display 120, the vital information which has no measurement error among the maximum value and the minimum value of the measured non-invasive blood pressure, and the arterial oxygen saturation “SpO₂” and the pulse rate “SpO₂-PR” of the patient. The display 120 displays, by numerical values, the vital information which has no measurement error, for example, the maximum value and the minimum value of the non-invasive blood pressure of the patient in accordance with the instruction of the display controller 106 (S206).

Next, the display 120 displays, with predetermined colors respectively, the tab portions of the vital information which has no measurement error provided on the numerical value display portions of the maximum value and the minimum value of the non-invasive blood pressure, and the arterial oxygen saturation “SpO₂” and the pulse rate “SpO₂-PR” of the patient in accordance with the instruction of the display controller 106. For example, the tab portion 134 provided on the numerical value display portion of the maximum value and the minimum value of the non-invasive blood pressure of the patient is displayed with a predetermined color (S207).

The display of the display 120 of the patient monitor device 100 when a part of the measurement is not completed is as illustrated in FIG. 11. For example, when an error occurs in the measurement of the arterial oxygen saturation “SpO₂” and the pulse rate “SpO₂-PR” of the patient, such as forgetting to measure or being unable to measure, the numerical value of the respective measured vital information is displayed on the respective display regions 130, and portions other than the characters of the tab portion 134 are colored respectively with predetermined colors and are lighted or blinked. Therefore, no numerical value is displayed on the numerical value display portion 142 of the arterial oxygen saturation “SpO₂” and the pulse rate “SpO₂-PR”, and although the characters “SpO₂” and “SpO₂-PR” are displayed on the tab portion 144, the tab portion 144 is not displayed with the predetermined color. Since the tab portions 134 and 144 are arranged in a line along the array of the respective numerical value display portions 132 and 142, when a part of the measurement of the vital information of the patient is not completed, the fact that a part of the measurement is not completed can be sensed by the medical worker at a glance since tabs of the part are not displayed with colors.

As described above in Embodiment 1 and Embodiment 2, according to the patient monitor device of the presently disclosed subject matter, when the measurement of the vital information of the patient is completed, the completion of the measurement is displayed on the tab portions arranged in a line along the array of the numerical value display portions in addition to the numerical values displayed on the numerical value display portions. Thus, the completion of the measurement of the vital information of the patient is displayed with high visibility. In particular, since the tab portions are arranged in a line, when the measurement of all of the vital information is completed, the tab portions of all the display regions simultaneously notify the completion of the measurement, so that the completion of the measurement can be recognized at a glance. Therefore, the medical worker can easily recognize a measurement failure of vital information, such as forgetting to measure or being unable to measure, at a glance.

A patient monitor device to achieve the object is configured to display vital information of a living body. The patient monitor device includes a display; and circuitry configured to individually display at least two types of the vital information of the living body on a plurality of display regions of the display. Each of the display regions includes: a numerical value display portion for displaying measured vital information of the living body as a numerical value; and a tab portion for notifying of completion of the measurement after the measurement of the vital information is completed, and tab portions of respective display regions are arranged in a line along an array of respective numerical value display portions.

According to the patient monitor device of the presently disclosed subject matter, when measurement of vital information of a patient is completed, the completion of the measurement is displayed on the tab portions arranged in a line along the array of the numerical value display portions in addition to the numerical values displayed on the numerical value display portions. Thus, the completion of the measurement of the vital information of the patient is displayed with high visibility. In particular, since the tab portions are arranged in a line, when the measurement of all of the vital information is completed, the tab portions of all the display regions simultaneously notify the completion of the measurement, and the completion of the measurement can be grasped sensually at a glance.

What is claimed is:

1. A patient monitor device configured to display vital information of a living body, comprising:
a display; and
circuitry configured to individually display at least two types of the vital information of the living body on a plurality of display regions of the display, wherein
each of the display regions includes: a numerical value display portion for displaying measured vital information of the living body as a numerical value; and a tab portion for notifying of completion of the measurement after the measurement of the vital information is completed, and
tab portions of respective display regions are arranged in a line along an array of respective numerical value display portions.

2. The patient monitor device according to claim 1, wherein the circuitry is configured to display the tab portion for notifying of the completion of the measurement by lighting or blinking after the measurement of the vital information is completed.

3. The patient monitor device according to claim 1, wherein
the tab portion includes a character for representing a type of the measured vital information, and
the circuitry is configured to cause the character to be lighted or blinked before the measurement of the vital information is completed, and to cause a portion other than the character in the tab portion to be lighted or blinked after the measurement of the vital information is completed.

4. The patient monitor device according to claim 1, wherein, in each display region, a color of a number displayed on the numerical value display portion and a color of the tab portion are a same color.

5. The patient monitor device according to claim 1, wherein the tab portion is provided to extend in a width direction of each display region.

6. The patient monitor device according to claim 1, wherein the circuitry is configured to change a display area of each display region depending on the number of the types of the measured vital information.

7. The patient monitor device according to claim 1, wherein the circuitry is configured to display all of the display regions in the display.
